(19) United States
(12) Patent Application Publication
(10) Pub. No.: US 2025/0325177 A1
(43) Pub. Date: Oct. 23, 2025

(54) OPTICAL COHERENCE TOMOGRAPHY SYSTEM AND SCANNING DEVICE THEREOF

(71) Applicant: Medimaging Integrated Solution, Inc., Hsinchu (TW)

(72) Inventors: CHU-MING CHENG, Hsinchu (TW); YU-TSUNG LEE, Hsinchu (TW); CHING LUNG LAI, Hsinchu (TW)

(21) Appl. No.: 19/095,554

(22) Filed: Mar. 31, 2025

(30) Foreign Application Priority Data
Apr. 22, 2024 (TW) 113114857

(51) Int. Cl. A61B 3/10; A61B 3/00; G02B 3/00; G02B 3/02; G02B 3/12

(52) U.S. Cl. CPC A61B 3/102; A61B 3/0008; G02B 3/0037; G02B 3/02; G02B 3/12; A61B 2560/0431; A61B 2562/228; A61B 2576/02

(57) ABSTRACT

A scanning device cooperates with a host machine to form an optical coherence tomography (OCT) system. The host machine outputs a sampling light. The scanning device includes, in order from the host machine to the eyeball, a collimating lens, a variable-focus liquid lens, a microelectromechanical system-based mirror (MEMS mirror), and a scanning lens assembly, which are optically coupled to each other. The present invention features compactness and is exempted from mounting/dismounting lenses via using the variable-focus liquid lens to compensate eyeballs for different diopters and using the scanning lens assembly to switch the scanned position (the anterior or posterior region of the eyeball). In addition, the distance from the MEMS mirror to the eyeball is fixed. In other words, no matter whether the scanning device is scanning the anterior or posterior region of the eyeball, there is no need to change the distance between the tester and the scanning system.

### BACKGROUND OF THE INVENTION

### 1. FIELD OF THE INVENTION

The present invention relates to an eyeball inspection device, particularly to an optical coherence tomography (OCT) system and a scanning device thereof.

### 2. DESCRIPTION OF THE PRIOR ART

The optical coherence tomography system is an indispensable apparatus in ophthalmology, used to perform tomography of the anterior and posterior region of an eyeball to aid in diagnosis of the eyeball.

In order to scan different regions of an eyeball, the optical coherence tomography system needs focus adjustment ability. The optical coherence tomography system also needs diopter compensation ability to adapt eyeballs having different diopters, such as eyeballs suffering myopia and hyperopia.

The conventional optical coherence tomography system can only scan a single region of the eyeball. The optical coherence tomography system able to scan the anterior region of eyeballs is unable to scan the posterior region of eyeballs. The optical coherence tomography system able to scan the posterior region of eyeballs is unable to scan the anterior region of eyeballs.

Thus, different lens modules need mounting and dismounting, and the distance between the tester and the system needs varying, whereby to enable the optical coherence tomography system to scan different regions.

Those operations increase the difficulty of using the system. Alternatively, a bulky and complicated optical coherence tomography system may be used to exempt the operator from mounting/dismounting lens modules and varying the distance between the tester and the system.

Therefore, the current optical coherence tomography system is hard to satisfy the requirements of easy operation and lightweight/compactness.

### SUMMARY OF THE INVENTION

The present invention proposes an optical coherence tomography

(OCT) system to provide an easy-to-operate and lightweight/compact eyeball inspection apparatus.

The present invention provides a scanning device, which cooperates with a host machine to form an optical coherence tomography system. The host machine outputs a sampling light. The scanning device comprises a collimating lens, a variable-focus liquid lens, a microelectromechanical system-based mirror (MEMS mirror), and a scanning lens assembly. The collimating lens is optically coupled to the host machine through optical fiber and used to collimate the sampling light. The variable-focus liquid lens is optically coupled to the collimating lens. The MEMS mirror is optically coupled to the various-focus liquid lens, so as to deflect the sampling light. The scanning lens assembly is optically coupled to the MEMS mirror to scan the anterior or posterior region of an eyeball with the sampling light. The distance between MEMS mirror and the eyeball is fixed.

Further, the present invention provides another scanning device, which cooperates with a host machine to form an optical coherence tomography system. The host machine outputs a sampling light. The scanning device comprises a collimating lens, a microelectromechanical system-based mirror (MEMS mirror), a variable-focus liquid lens, and a scanning lens assembly. The collimating lens is optically coupled to the host machine through optical fiber and used to collimate the sampling light. The MEMS mirror is optically coupled to the various-focus liquid lens, so as to deflect the sampling light. The variable-focus liquid lens is optically coupled to the MEMS mirror. The scanning lens assembly is optically coupled to the various-focus liquid lens, whereby the sampling light can scan the anterior or posterior region of an eyeball. The distance between MEMS mirror and the eyeball is fixed.

In some embodiments, the scanning lens assembly is moved along an optical axis to adjust the focal length and enable the sampling light to scan the anterior or posterior region of the eyeball,

In some embodiments, the scanning lens assembly includes at least one aspherical lens.

In some embodiments, the scanning lens assembly includes a first lens group, a second lens group, and a third lens group in sequence from the position near the eyeball to the position away from the eyeball. The effective focal length of the first lens group is a positive value. The effective focal length of the second lens group is a negative value. The effective focal length of the third lens group is a positive value.

In some embodiments, the effective focal length of the first lens group ranges within 15-30 mm.

In some embodiments, the first lens group is formed by a first lens whose two surfaces are convex. The second lens group is formed by a second lens whose two surfaces are concave. The third lens group is formed by three third lenses.

In some embodiments, one of the outer diameters of the first lens group, which is near the eyeball mostly, is D1. D1 ranges within 20-40 mm.

In some embodiments, the input voltage varies the focal length of the variable-focus liquid lens to make the sampling light able to scan eyeballs having different diopters.

The present invention also provides an optical coherence tomography system, which comprises a host machine, a scanning light source, a fiber coupler, a light attenuator, a reference light polarization controller, a reference light device, a spectrometer, and a scanning device. The scanning light source outputs a light source. The fiber coupler is optically coupled to the scanning light source and splits the light source into a reference light and a sampling light. The light attenuator is optically coupled to the fiber coupler and used to modify the intensity of the reference light. The reference light polarization controller is optically coupled to the light attenuator and used to polarize the reference light. The reference light device includes a reference light collimating lens, a chromatic dispersion compensation lens, a focusing lens, and a reference light reflecting mirror. The reference light passes through the reference light collimating lens, the chromatic dispersion compensation lens and the focusing lens, reflected by the reference light reflecting mirror and then coming back along the original optical path to the fiber coupler. The scanning device is optically coupled to the fiber coupler. The structure of the scanning device has been described above. The sampling light is reflected by the eyeball and returns along the original optical path to the fiber coupler. The spectrometer receives the reflected reference light and the reflected sampling light to generate a light signal.

In some embodiments, the optical coherence tomography system further comprises a computer. The computer receives the light signal and performs computation to reconstruct a tomography image.

In some embodiments, the spectrometer includes a diffraction grating and a linear scanning camera.

In some embodiments, the scanning light source includes a superluminescent diode.

In some embodiments, the optical coherence tomography system further comprises a handheld casing. The scanning device is disposed inside the handheld casing and optically coupled to the host machine through optical fiber.

It is learned from the above description: the present invention uses the variable-focus liquid lens to compensate for the eyeballs having different diopters and uses the scanning lens assembly to change the scanned position and determine whether the scanned position is the anterior or posterior region of the eyeball. The present invention features lightweight and compactness and is exempted from mounting/dismounting lenses. The present invention has the advantage: the distance between the MEMS mirror and the eyeball is fixed. It means: the distance between the scanning system and the tester needn't change no matter whether the scanning device scans the anterior or posterior region of the eyeball. Thereby, the operation becomes easier, and the objective of the present invention is indeed achieved.

The objective, technologies, features and advantages of the present invention will become apparent from the following description in conjunction with the accompanying drawings wherein certain embodiments of the present invention are set forth by way of illustration and example.

### BRIEF DESCRIPTION OF THE DRAWINGS

The foregoing conceptions and their accompanying advantages of this invention will become more readily appreciated after being better understood by referring to the following detailed description, in conjunction with the accompanying drawings, wherein:

FIG. 1 is a diagram schematically showing an optical coherence tomography system according to one embodiment of the present invention;

FIG. 2 is a diagram schematically showing a scanning device according to one embodiment of the present invention;

FIGS. 3A-3D are diagrams schematically showing a scanning device according to one embodiment of the present invention;

FIG. 4 is an OCT image of a posterior region of an eyeball, which is obtained by the present invention;

FIG. 5 is an OCT image of an anterior region of an eyeball, which is obtained by the present invention; and

FIG. 6 is a diagram schematically showing a scanning device according to one embodiment of the present invention.

### DESCRIPTION OF THE PREFERRED EMBODIMENTS

Various embodiments of the present invention will be described in detail below and illustrated in conjunction with the accompanying drawings. In addition to these detailed descriptions, the present invention can be widely implemented in other embodiments, and apparent alternations, modifications and equivalent changes of any mentioned embodiments are all included within the scope of the present invention and based on the scope of the Claims. In the descriptions of the specification, in order to make readers have a more complete understanding about the present invention, many specific details are provided; however, the present invention may be implemented without parts of or all the specific details.

In addition, the well-known steps or elements are not described in detail, in order to avoid unnecessary limitations to the present invention. Same or similar elements in Figures will be indicated by same or similar reference numbers. It is noted that the Figures are schematic and may not represent the actual size or number of the elements. For clearness of the Figures, some details may not be fully depicted.

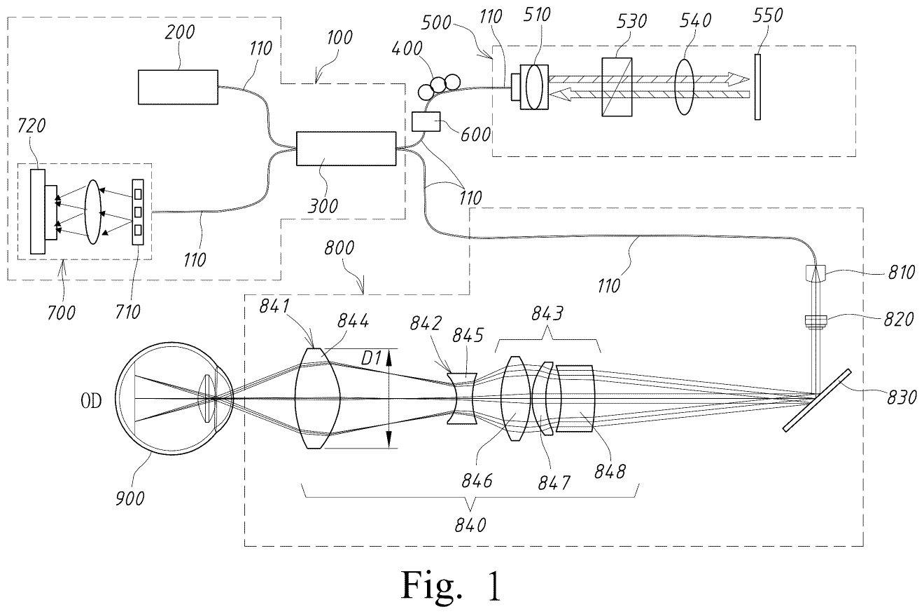

Refer to FIG. 1. In some embodiments, the optical coherence tomography system of the present invention comprises a host machine 100, a scanning light source 200, a fiber coupler 300, a reference light polarization controller 400, a reference light device 500, a spectrometer 700, and a scanning device 800.

The scanning light source 200 is disposed inside the host machine 100 and outputs a light source. The scanning light source 200 has a superluminescent diode. The superluminescent diode has high output power and a broad band spectrum, suitable to be used in ophthalmological inspection instruments.

The fiber coupler 300 is optically coupled to the scanning light source 200 in the host machine 100. The fiber coupler 300 is used to split the light source into a reference light and a sampling light, whereby two optical paths are formed. One of them is a reference light optical path; another one of them is a sampling light optical path.

The reference light optical path passes through a light attenuator 600, the reference light polarization controller 400 and the reference light device 500. The light attenuator 600 is optically coupled to the fiber coupler 300, used to adjust the intensity of the reference light. The reference light polarization controller 400 is optically coupled to the light attenuator 600, used to polarize the reference light. The reference light device 500 includes a reference light collimating lens 510, a chromatic dispersion compensation lens 530, a focusing lens 540, and a reference light reflecting mirror 550. The reference light passes through the reference light collimating lens 510, the chromatic dispersion compensation lens 530 and the focusing lens 540 and is reflected by the reference light reflecting mirror 550. Next, the reflected reference light returns to the fiber coupler 300 along the same reference light optical path. Next, the reflected reference light passes through the fiber coupler 300 and then reaches the spectrometer 700.

The sampling light optical path passes through the scanning device 800 and then reaches an eyeball 900. The structure of the scanning device 800 will be described in details below. The sampling light is reflected by the eyeball 900 and then returns to the fiber coupler 300 along the same sampling light optical path.

The spectrometer 700 is optically coupled to the fiber coupler 300. The spectrometer 700 includes a diffraction grating 710 and a linear scanning camera 720, which are optically coupled to each other. The spectrometer 700 receives the reflected reference light and the reflected sampling light and performs computation to generate tomography images.

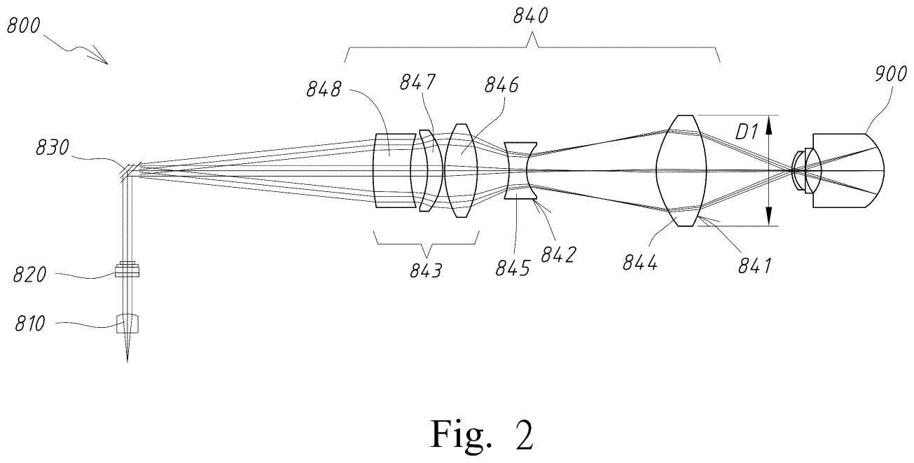

Refer to FIG. 2. The scanning device 800 is described in details herein. The scanning device 800 and the host machine 100 form the optical coherence tomography system. The host machine 100 outputs a sampling light. The scanning device 800 includes a collimating lens 810, a variable-focus liquid lens 820, a microelectromechanical system-based mirror (MEMS mirror) 830, and a scanning lens assembly 840.

The collimating lens 810 is optically coupled to the host machine 100 through optical fiber 110 and used to collimate the sampling light. The variable-focus liquid lens 820 is optically coupled to the collimating lens 810. The MEMS mirror 830 is optically coupled to the variable-focus liquid lens 820 and used to deflect the sampling light. The scanning lens assembly 840 is optically coupled to the MEMS mirror 830 to make the sampling light scan the anterior or posterior region of the eyeball 900. The distance between the MEMS mirror 830 and the eyeball 900 is fixed.

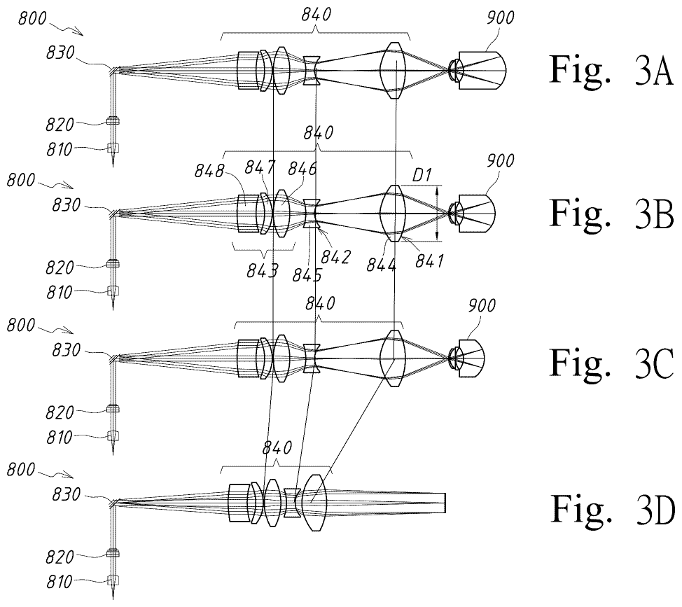

Refer to FIGS. 3A-3D. The scanning device 800 may be switched between an anterior segment (AS) mode and a posterior segment (PS) mode. In the PS mode (shown in FIGS. 3A-3C), the sampling light scans the posterior region of the eyeball 900. In the AS mode (shown in FIG. 3D), the sampling light scans the anterior region of the eyeball 900.

Refer to FIGS. 3A-3C again. The eyeball 900 shown in FIG. 3A suffers myopia. The eyeball 900 shown in FIG. 3B is a normal one. The eyeball 900 shown in FIG. 3C suffers hyperopia. The eyeballs 900 of myopia and hyperopia respectively have different diopters. Considering the difference of diopters, the focal length of the variable-focus liquid lens 820 needs to be changed to compensate for different diopters. Varying the input voltage changes the focal length of the variable-focus liquid lens 820 and thus makes the sampling light able to scan the eyeballs having different diopters. Thereby, the sampling light can scan the posterior region correctly.

Refer to FIGS. 3A-3D again. The scanning lens assembly 840 may be moved along the optical axis to adjust the focal length and enable the sampling light to scan the anterior or posterior region of the eyeball. While the system is switched from the PS (posterior segment) mode to the AS (anterior segment) mode, the sampling light is enabled to scan the anterior region of the eyeball via varying the distance between the scanning lens assembly 840 and the MEMS mirror 830 and via varying the distances between different lenses inside the scanning lens assembly 840 simultaneously.

It is learned from the above description: the present invention compensates for different diopters of the eyeballs 900 using the variable-focus liquid lens 820 and switches the scanned position between the anterior region and the posterior region using the scanning lens assembly 840. Thereby, the present invention is lightweight/compact and exempted from mounting/dismounting lenses. The present invention has an advantage of having a fixed distance between the MEMS mirror 830 and the eyeball 900. In other words, no matter whether the scanning device 800 is to scan the anterior or posterior region of the eyeball 900, it is unnecessary to change the distance between the tester and the optical coherence tomography system. Thereby, the operation becomes easier. Therefore, the objective of the present invention is indeed achieved.

In some embodiments, the optical coherence tomography system further comprises a handheld casing. The scanning device 800 is disposed inside the handheld casing. The scanning device 800 is optically coupled to the host machine 100 through optical fiber 110. One end of the optical fiber 110 is connected with the fiber coupler 300; another end of the optical fiber 110 is connected with the collimating lens 810 of the scanning device 800. The optical fiber 110 is also used to connect the scanning light source 200, the fiber coupler 300, the light attenuator 600, the reference light polarization controller 400 and the reference light device 500. Further, the optical fiber 110 is also used to connect the fiber coupler 300 and the spectrometer 700.

In some embodiments, the scanning lens assembly 840 includes at least one aspherical lens.

Refer to FIG. 2. In some embodiments, the scanning lens assembly 840 includes a first lens group 841, a second lens group 842, and a third lens group 843 in sequence from the position near the eyeball 900 to the position away from the eyeball 900. The effective focal length of the first lens group 841 is a positive value. The effective focal length of the second lens group 842 is a negative value. The effective focal length of the third lens group 843 is a positive value. In details, the effective focal length of the first lens group 841 ranges within 15-30 mm. Such a range of focal length is favorable to realize an optical coherence tomography system, which is lightweight/compact and easy to be held and operated by the hand.

In some embodiments, the first lens group 841 is formed by a first lens 844 whose two surfaces are convex. The second lens group 842 is formed by a second lens 845 whose two surfaces are concave. The third lens group 843 is formed by three third lenses 846, 847 and 848. While the system is switched from the PS (posterior segment) mode to the AS (anterior segment) mode, the distances from the MEMS mirror 830 to first lens 844, the second lens 845 and the third lenses 846, 847 and 848 may be varied; the distances between the third lenses 846, 847 and 848 are essentially kept unchanged.

In some embodiments, one of the outer diameters of the first lens

group 841, which is near the eyeball mostly, is D1. D1 ranges within 20-40 mm. Such an outer diameter range allows sufficient sampling light to pass and favors lightweight and compactness.

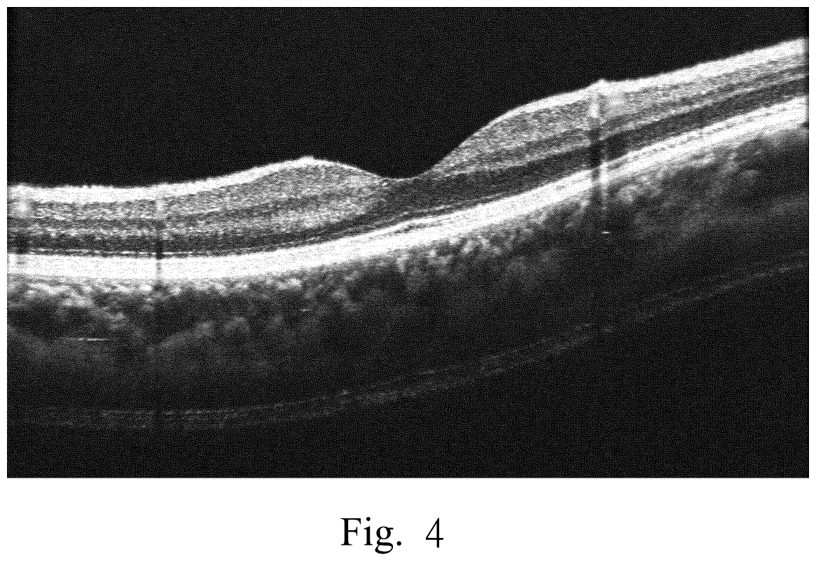

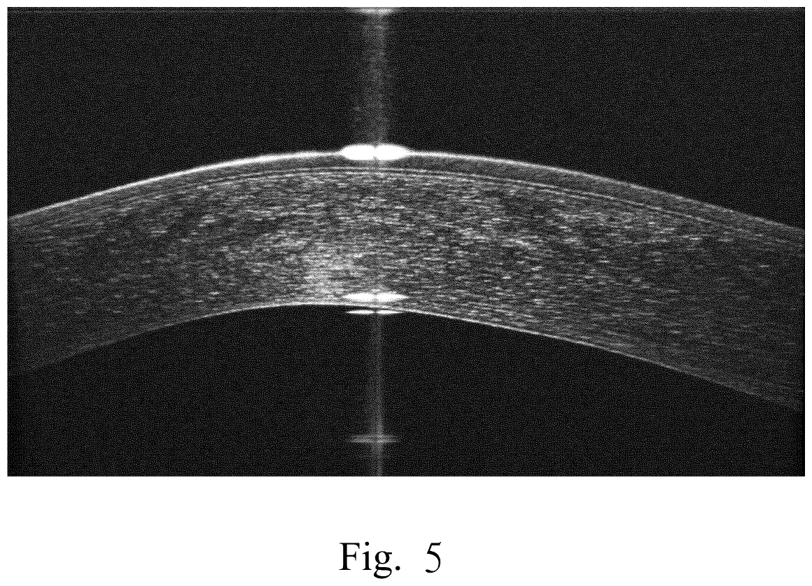

In some embodiments, the optical coherence tomography system further comprises a computer. The computer receives a light signal from the spectrometer 700 and performs computation to reconstruct tomography images. In details, an optical sensor receives the light signal and converts the light signal into an electric signal; an analog-to-digital converter converts the electric signal into a digital signal; the computer receives the light signal-related digital signal and performs computation to work out tomography images. Refer to FIG. 4 and FIG. 5 for the results of tomography scanning. FIG. 4 shows a tomography image of the posterior region of an eyeball. FIG. 5 shows a tomography image of the anterior region of an eyeball.

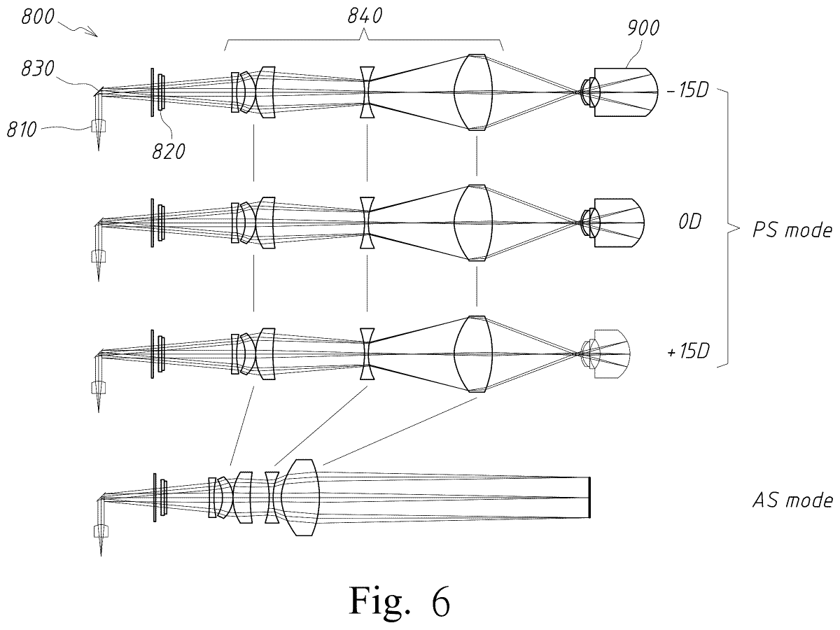

Refer to FIG. 6. Some embodiments are essentially the same as the aforementioned embodiments but are different from the aforementioned embodiments in that the variable-focus liquid lens 820 is disposed between the MEMS mirror 830 and the scanning lens assembly 840. In details, the scanning device 800 and the host machine 100 form the optical coherence tomography system; the host machine 100 outputs a sampling light; the scanning device 800 includes a collimating lens 810, a variable-focus liquid lens 820, a microelectromechanical system-based mirror (MEMS mirror) 830, and a scanning lens assembly 840; the collimating lens 810 is optically coupled to the host machine 100 through optical fiber 110 and used to collimate the sampling light; the MEMS mirror 830 is optically coupled to the collimating lens 810 and used to deflect the sampling light; the variable-focus liquid lens 820 is optically coupled to the MEMS mirror 830; the scanning lens assembly 840 is optically coupled to the variable-focus liquid lens 820 and used to make the sampling light scan the anterior or posterior region of the eyeball 900; the distance between the MEMS mirror 830 and the eyeball 900 is fixed.

In conclusion, the present invention has the following advantages:
1. While the scanning device 800 is to scan the anterior or posterior region of the eyeball 900, it is unnecessary to mount/dismount lenses. The scanning mode is switched to scan the anterior or posterior region of the eyeball 900 via changing the relative distance between the scanning lens assembly 840 and the MEMS mirror 830. Therefore, the present invention can be operated easily.
2. The distance between the MEMS mirror 830 and the eyeball 900 is constant. The tester needn't adjust the distance between the tester and the host machine 100/the handheld casing. Therefore, the present invention can be operated conveniently.
3. The scanning device 800 has simpler and fewer components. Therefore, the present invention is lightweight and compact and favorable to be held by the hand for operation.
4. The variable-focus liquid lens 820 can shorten the time of focusing and accelerate inspection.
5. The scanning device 800 has simpler and fewer components. Therefore, the present invention can achieve optical collimation easily.

While the invention is susceptible to various modifications and alternative forms, a specific example thereof has been shown in the drawings and is herein described in detail. It should be understood, however, that the invention is not to be limited to the particular form disclosed, but to the contrary, the invention is to cover all modifications, equivalents, and alternatives falling within the appended claims.

1. A scanning device, which cooperates with a host machine to form an optical coherence tomography system, wherein the host machine outputs a sampling light, and the scanning device comprises
a collimating lens, optically coupled to the host machine through optical fiber, and collimating the sampling light;
a variable-focus liquid lens, optically coupled to the collimating lens;
a microelectromechanical system-based mirror (MEMS mirror), optically coupled to the variable-focus liquid lens, and deflecting the sampling light; and
a scanning lens assembly, optically coupled to the MEMS mirror, and making the sampling light scan an anterior or a posterior region of an eyeball,
wherein a distance between the MEMS mirror and the eyeball is fixed.

2. The scanning device according to claim 1, wherein the scanning lens assembly is moved along an optical axis to adjust a focal length and make the sampling light scan the anterior or posterior region.

3. The scanning device according to claim 1, wherein the scanning lens assembly includes at least one aspherical lens.

4. The scanning device according to claim 1, wherein the scanning lens assembly includes a first lens group, a second lens group, and a third lens group in sequence from a position near the eyeball to a position away from the eyeball; an effective focal length of the first lens group is a positive value; an effective focal length of the second lens group is a negative value; an effective focal length of the third lens group is a positive value.

5. The scanning device according to claim 4, wherein the effective focal length of the first lens group ranges within 15-30 mm.

6. The scanning device according to claim 4, wherein the first lens group is formed by a first lens whose two surfaces are convex; the second lens group is formed by a second lens whose two surfaces are concave; the third lens group is formed by three third lenses.

7. The scanning device according to claim 4, wherein one of outer diameters of the first lens group, which is near the eyeball mostly, is D1; D1 ranges within 20-40 mm.

8. The scanning device according to claim 1, wherein varying an input voltage changes a focal length of the variable-focus liquid lens and makes the scanning lens assembly able to use the sampling light to scan eyeballs having different diopters.

9. A scanning device, which cooperates with a host machine to form an optical coherence tomography system, wherein the host machine outputs a sampling light, and the scanning device comprises
a collimating lens, optically coupled to the host machine through optical fiber, and collimating the sampling light;
a microelectromechanical system-based mirror (MEMS mirror), optically coupled to the collimating lens, and deflecting the sampling light;
a variable-focus liquid lens, optically coupled to the MEMS mirror; and
a scanning lens assembly, optically coupled to the variable-focus liquid lens, and making the sampling light scan an anterior or posterior region of an eyeball,
wherein a distance between the MEMS mirror and the eyeball is fixed.

10. The scanning device according to claim 9, wherein the scanning lens assembly is moved along an optical axis to adjust a focal length and make the sampling light scan the anterior or posterior region.

11. The scanning device according to claim 9, wherein the scanning lens assembly includes at least one aspherical lens.

12. The scanning device according to claim 9, wherein the scanning lens assembly includes a first lens group, a second lens group, and a third lens group in sequence from a position near the eyeball to a position away from the eyeball; an effective focal length of the first lens group is a positive value; an effective focal length of the second lens group is a negative value; an effective focal length of the third lens group is a positive value.

13. The scanning device according to claim 12, wherein the effective focal length of the first lens group ranges within 15-30 mm.

14. The scanning device according to claim 12, wherein the first lens group is formed by a first lens whose two surfaces are convex; the second lens group is formed by a second lens whose two surfaces are concave; the third lens group is formed by three third lenses.

15. The scanning device according to claim 12, wherein one of outer diameters of the first lens group, which is near the eyeball mostly, is D1; D1 ranges within 20-40 mm.

16. The scanning device according to claim 9, wherein varying an input voltage changes a focal length of the variable-focus liquid lens and makes the scanning lens assembly able to use the sampling light to scan the eyeballs having different diopters.

17. An optical coherence tomography system, comprising
a host machine, including a scanning light source, a fiber coupler, and a spectrometer, wherein the scanning light source outputs a light source; the fiber coupler is optically coupled to the scanning light source and splits the light source into a reference light and a sampling light;
the spectrometer is optically coupled to the fiber coupler;
a light attenuator, optically coupled to the optical coupler and used to modify an intensity of the reference light;
a reference light polarization controller, optically coupled to the light attenuator and used to polarize the reference light;
a reference light device, including a reference light collimating lens, a chromatic dispersion compensation lens, a focusing lens, and a reference light reflecting mirror, wherein the reference light passes through the reference light collimating lens, the chromatic dispersion compensation lens and the focusing lens and is reflected by the reference light reflecting mirror to return to the fiber coupler along an original optical path; and
a scanning device according to claim 1, optically coupled to the fiber coupler, wherein the sampling light passes through the scanning device to reach an eyeball and is reflected by the eyeball to return to the fiber coupler through the original optical path,
wherein the spectrometer receives the reference light and the sampling light, which are reflected by the eyeball, to generate a light signal.

18. The optical coherence tomography system according to claim 17, further comprising a computer, wherein the computer performs computation according to the light signal to reconstruct a tomography image.

19. The optical coherence tomography system according to claim 17, wherein the spectrometer includes a diffraction grating and a linear scanning camera.

20. The optical coherence tomography system according to claim 17, wherein the scanning light source has a superluminescent diode.

21. The optical coherence tomography system according to claim 17, further comprising a handheld casing, wherein the scanning device is disposed inside the handheld casing and optically coupled to the host machine through optical fiber.
